(19) United States
(12) Patent Application Publication
(10) Pub. No.: US 2004/0073318 A1
(43) Pub. Date: Apr. 15, 2004

(54) MEDICAL IMPLANTS MADE OF MOULDINGS

(76) Inventor: JOHANNES REINMULLER, WIESBADEN (DE)

Correspondence Address: FULBRIGHT AND JAWORSKI, 666 FIFTH AVE, NEW YORK, NY, 10103, US

(21) Appl. No.: 08/732,408

(22) PCT Filed: Apr. 12, 1995
(86) PCT No.: PCT/EP95/01357

(30) Foreign Application Priority Data
Apr. 22, 1994 (DE) P 44 14 103.3

(51) Int. Cl. A61F 2/12; A61F 2/02

(52) U.S. Cl. 623/23.72; 623/11.11; 623/8

(57) ABSTRACT

An implant for medical purposes based on a physiologically compatible plastic is composed of thin foldable structural elements having a surface that can be wetted by a fluid lubricant and is preferably surrounded by a covering that contains a lubricant such as e.g. an aqueous fluid.

This invention concerns plastic implants for medical purposes and in particular implants composed of thin foldable structural elements.

In human and veterinary medicine congenital or acquired organ or soft-tissue defects are filled using plastic implants (PI) which are operatively introduced into the organism. In contrast to orthopaedic implants or to artificial organs which are also implant ed in the organism, the object of the aforementioned plastic implants is to provide volume substitution of soft-tissues and in a particular embodiment to effect stretching and distension of tissue especially of skin tissue.

Typical examples of PI include the substitution of missing mammary tissue by silicone pads which are implanted in front of or behind the pectoral muscle or implants to substitute missing testicles. Examples of implants for stretching and distending tissue to construct soft parts in the face, at the extremities or on the torso are conventional expanders such as e.g. the Becker expander as described by Joseph G. McCarthy, Plastic Surgery (1990), W. B. Sounders Comp., Volume 1, chapter 13, p. 486.

In order to achieve a reconstruction of soft tissues which looks as natural as possible materials are preferably used for plastic implants whose mechanical properties are very close to those of natural tissues. In addition it is required that the implant material used is physiologically compatible, does not have toxic effects and has an extreme chemical and mechanical long-term stability. In general elastically deformable plastics fulfil these requirements especially in the form of gels. The use of dimethylsiloxane polymers (PDMS) have proven to be particularly advantageous due to their excellent chemical stability.

Conventional implants comprise an outer covering made of silicone material into the interior of which a high-polymer gel e.g. a PDMS gel is introduced or aqueous solutions. The gel or aqueous solution can be added before the implantation or in the case of an aqueous solution subsequent to implantation using a valve which is accessible from the body surface. Such implants have for example been disclosed in U.S. Pat. Nos 4,428,364, 4,474,780, 4,671,255 and 4,840,615.

However, conventional products which use gels and especially products which use PDMS gels have the disadvantage that when the outer covering is damaged, e.g. in an accident, the surrounding tissue is infiltrated with (PDMS) gel under high pressure. Afterwards such a situation can hardly be brought under control by surgical measures. Moreover, recent investigations have raised doubts about the physiological compatibility of the PDMS implants. It is assumed that lower-molecular components of PDMS gels diffuse through the outer covering and thus reach the surrounding tissue causing long-term irreversible injury to health. Although, this penetration can be reduced by introducing barrier layers, it cannot be avoided completely.

Attempts to replace PDMS gels by other materials due to the aforementioned disadvantages have not yet led to a satisfactory result:

Physiological saline solutions are not suitable since as a filler material they give a plastic implant undesired mechanical and compression elasticity properties. In addition such implants lose their liquid volume within a few years since the outer covering made of PDMS rubber is not a barrier for lower molecular substances.

The use of other substitutes such as e.g. hydrogels, dextrans, hylanes, gelatins or polyvinyl-pyrolidone (PVP) is disadvantageous due to their poor long-term stability or is limited due to their toxicity.

The object of the present invention was therefore to provide a plastic implant which ensures the advantages of PDMS gels i.e. compression elasticity and thus tissue-like properties as well as chemical long-term stability but at the same time does not have the aforementioned disadvantages i.e. diffusion of lower molecular toxic components into the surrounding tissue and infiltration of the surrounding tissue with PDMS gel when the outer covering is damaged.

This object is achieved according to the present invention by an implant for medical purposes based on a physiologically compatible plastic which is composed of thin foldable structural elements with a surface that can be wetted by a fluid lubricant.

The volume of the implant is in this case usually determined by the thin structural elements whereas the volume of lubricant is not as important. In order to construct an implant according to the invention thin foils are for example used as the structural elements wherein numerous layers lying on top of one another yield an implant of the desired thickness.

The desired tissue-like elastic properties of the implant of the present invention are essentially achieved by the ability to displace adjacent layers of the thin foldable structural elements, the sliding effect being mediated and facilitated by a suitable lubricant.

The structural elements of the present invention can either be introduced without an outer covering directly into a pre-formed implant pocket in the tissue or, like conventional implants, they can be surrounded by an outer covering in which case the gels of conventional implants are replaced by structural elements of the present invention.

All physiologically compatible plastics come into consideration as materials for the structural elements. The term “physiologically compatible plastics” in this application generally means naturally occurring and artificially produced materials or biomaterials that are physiologically compatible. In a particular embodiment the structural elements are composed of silicone rubber, silicone rubber made of polydimethylsiloxane being preferred. In a further embodiment structural elements made of polysaccharides are used in which case structural elements made of cuprophane foil represent a preferred embodiment. However, the invention is not limited to the aforementioned materials and other physiologically compatible plastics and biomaterials which can be processed to form thin structural elements are part of the present invention.

The thin foldable structural elements of the invention can be provided in various embodiments. It is preferable to use a foil which is folded in a suitable manner or to use several layers of foil which lie on top of one another and are cut to make the shape of the final implant. It has been found that it is advantageous to use foils having a thickness of about 5 to about 500 μm, 10 to 200 μm being preferred and 30 to 70 μm being especially preferred. In a further embodiment of the invention the structural elements are provided in the form of capillary tubes or in a tubular form. It is also possible to use the structural elements in a strand form (“silicone spaghetti”). In this connection it is possible to achieve better results using smaller strand thicknesses with regard to the tissue-like properties of the implants. However, it is preferred for technical reasons to use strand thicknesses between 0.1 and 1 mm.

The present invention is not limited to the aforementioned embodiments and other embodiments such as for example loose open-pored plastic foams are also a subject matter of this invention. In some cases it may be particularly expedient to use a combination of two or several embodiments. Such combinations are also encompassed by the present invention.

The implants of the invention can be monitored post-operatively after introduction into the tissue. PDMS foils are for example more dense than tissue towards X-ray radiation which enables the position of an implant to be monitored postoperatively by means of an X-ray examination. Implants according to the invention which comprise structural elements made of PDMS are thus accessible to a postoperative examination without further additives. In other cases the addition of suitable materials that can be detected from the outside such as for example X-ray contrast agents in the plastic enable the implants to be monitored later in a simple manner.

The tissue-like properties of the implants of the present invention are mainly based on the sliding ability of adjacent layers of the thin structural elements, the sliding ability being facilitated by a suitable fluid lubricant.

The term “fluid lubricant” according to this application is in general understood as liquid and fluid phases which are physiologically compatible and which enable or/and improve the movement of surfaces of adjacent structural elements. The type of lubricant used only depends on the surface of the structural element used. In the case of a hydrophilic surface of the structural elements a hydrophilic fluid is also used as a lubricant. Sliding mediated by hydrophilic interactions is the preferred embodiment in which case it is especially preferable to use an aqueous liquid as the lubricant. Accordingly the sliding ability can also be achieved by using hydrophobic surfaces/lubricants, fats and oils being in this case preferred as hydrophobic lubricants. Sliding processes mediated hydrophilically/hydrophobically e.g. by liposomes or micelles of ampholytic substances are also a subject matter of this invention.

In many cases it can be advantageous to adapt the surfaces of the thin structural elements to the properties of the lubricant used by hydrophobization or hydrophilization in order to further improve their sliding ability. Corresponding processes are well-known to a person skilled in the art. The hydrophilization of silicone surfaces is for example described in DE-OS 4216271 by etching with a solution of KOH in methanol. EP-A-0 086 186 describes the hydrophilization of surfaces containing amines with substances that contain 2-amino-2-deoxyglycopyranosil residues. Suitable processes for hydrophobization for example include applying silicone layers or waxes.

The use of structural elements with hydrophilized surfaces represents a particularly advantageous application of the structural elements of the present invention. The hydrophilization binds the lubricant, preferably water, inside the implants and thus effectively prevents loss of volume by diffusion as occurs in conventional implants based on liquids. In this embodiment the implants of the present invention can optionally be implanted without addition of lubricant. In this case the lubricant is provided by the surrounding tissue since tissue fluid can diffuse into the inside of the implants and if present through the outer covering.

In a further preferred embodiment the implants of the present invention contain swellable materials. Suitable swellable materials for producing structural elements according to the invention are in general all physiologically compatible substances which can swell on contact with a suitable liquid. It is particularly advantageous to use polysaccharides, the polysaccharide cuprophane foil being particularly preferred. In this case water is the swelling as well as lubricating agent.

However, the use of swellable materials is not limited to the structural elements but rather a swellable lubricant can be used instead of or in addition to swellable structural elements. Suitable swellable lubricants are for example anhydrous polysaccharides, glycosaminoglycans, dextrans and such like. Hyaluronic acid is preferred. Further suitable swellable materials are known to a person skilled in the art.

In addition the implants of the present invention contain other additives such as dyes, antioxidants, stabilizing agents, disinfectants, antibiotics, salts and such like as needed or depending on the intended use.

The implants of the present invention are either used as soft tissue substitutes or to expand surrounding tissue. The plastic implants are usually surrounded by an outer elas tic co vering, the form of the impl ants being determined by the dimensions of the outer covering. Taut elastic properties can be achieved to a greater or lesser extent by overfilling or underfilling the outer covering with the structural elements according to the invention. The lubricant usually only fills the capillary gaps which occur between the layers of the filling material and thus makes only a slight contribution to the filling volume. As an alternative an implant according to the invention can be underfilled with swellable structural elements and/or lubricant in which case the final volume of the implant is determined in advance by the fill quantity and the give n swellability of the materials but is only achieved after implantation.

A particular type of application of the implants of the present invention is as so-called tissue expanders which are only subsequently brought to their final volume after implantation or incorporation of the expander. In this case a change in volume is regulated by addition or removal of lubricant. Similarly to the known Becker expander it is possible to remove the valve mechanism after the desired expander volume has been achieved by which means the expander containing the filling according to the invention becomes a permanent implant.

The advantages of the present invention are essentially that the thin foldable structural elements which represent the filling material of the implants according to the invention have the same long-term stability as the conventional outer covering material. The release of toxic substances over a long time period is considerably reduced by this. In addition the filling material is not dispersed into the surrounding tissue in an uncontrolled manner as is the case with conventional PDMS gels when the outer covering is damaged but can be removed surgically in a complete and simple manner. Released lubricant is taken up by surrounding tissue or mixes with tissue fluid without detrimental effects for the organism. In a preferred embodiment a great advantage is the simple sterilizability in an autoclave when using structural elements made of hydrophilized PDMS foil and water as the lubricant.

The present invention is illustrated by the following examples.

### EXAMPLE 1

Hydrophilization of Silicone Surfaces

Silicone rubber foils with a thickness of 0.02 mm, 0.2 mm and 0.3 mm and strand-like silicone rubber (silicone spaghetti) are hydrophilized by etching with a solution of 5.7 g KOH in 100 ml methanol as described in DE-OS 4216271.

### EXAMPLE 2

Mammary Implant

A covering of silicone rubber is produced with an elliptical cross-section and a circular longitudinal section with a wall thickness of 0.2 mm. Hydrophilized silicone rubber foil of thickness 0.02 mm from example 1 is introduced through an opening. The outer covering has a capacity of 200 ml in the relaxed state. The amount of added filling material corresponds to a volume of 190 ml. A volume of 10 ml water is also added to the interior. The filling aperture is tightly closed. The mammary implant produced in this way is degassed in a vacuum chamber and afterwards sterilized in an autoclave.

### EXAMPLE 3

Lip Implant

A length of hydrophilized silicone rubber foil from example 1 with a thickness of 0.02 mm and 4 cm in length and 6 cm in width is rolled up to form a round body. This round body is pulled into the upper lip with the aid of an awl through small incisions in the area of the angle of the mouth. The incisions are closed by stitches.

### EXAMPLE 4

Gluteal Implant

A silicone rubber covering is produced with an oval shape and a wall thickness of 0.3 mm and capacity of 180 ml. Hydrophilized silicone rubber from example 1 is introduced through an opening as round material with a diameter of 1 mm in an amount which corresponds to a volume of 170 ml. 8 ml water are additionally added. The covering is tightly sealed. The implant is degassed and autoclaved.

### EXAMPLE 5

Skin Expander

A silicone rubber covering is produced in a rectangular form with rounded corners and edges. It is hydrophilized internally and externally as in example 1. Foil material made from cellulose acetate with a thickness of 0.01 mm is added through an opening. A powder made of anhydrous cross-linked hyaluronic acid is interspersed between the individual layers of the foil. The covering is tightly sealed and the implant is autoclaved. After implanting the implant prepared in this way into the organism, for example into subcutaneous tissue, water penetrates into the interior through the silicone rubber covering and leads to a swelling of the filling material. The implant expands spatially and in this process expands the skin. The time-course of the increase in size is regulated by the inflow of water. The final volume of the skin expander is defined in advance on the basis of the swellability of the filling material by the amount of filler introduced.

1. Implant for medical purposed based on a physiologically compatible plastic,
wherein
it is composed of thin foldable structural elements having a surface that is wettable by a fluid lubricant.

2. Implant as claimed in claim 1,
wherein
it is surrounded by a covering.

3. Implant as claimed in claim 1 or 2,
wherein
it has the shape of a foil with a thickness of 10 to 200 μm.

4. Implant as claimed in claim 1 or 2,
wherein
it has a strand form with a thickness of 0.1 to 1 mm per strand.

5. Implant as claimed in claim 1 or 2,
wherein
it is composed of tubular foils.

6. Implant as claimed in one of the claims 1 to 5,
wherein
it has a hydrophilized surface.

7. Implant as claimed in claim 6,
wherein
it contains an aqueous fluid as the lubricant.

8. Implant as claimed in one of the previous claims,
wherein
the thin foldable structural element is capable of swelling when contacted with a suitable liquid.

9. Implant as claimed in one of the previous claims,
wherein
the lubricant is swellable.

10. Implant as claimed in claim 9,
wherein
the lubricant is a polysaccharide or glucosaminoglycan.

11. Implant as claimed in one of the claims 1 to 5,
wherein
the thin foldable structural element has a hydrophobized surface.

12. Implant as claimed in claim 11,
wherein
it contains fat or oil as the fluid lubricant.

13. Implant as claimed in one of the previous claims,
wherein
the plastic is a silicone rubber.

14. Implant as claimed in claim 13,
wherein
the silicone rubber is composed of polydimethylsiloxane.

15. Implant as claimed in one of the claims 1 to 10,
wherein
the plastic is a polysaccharide.

16. Implant as claimed in claim 8, 9 and 15,
wherein
the polysaccharide is cuprophane foil.

17. Implant as claimed in one of the previous claims,
wherein
the foldable structural element has a foam structure.

18. Implant as claimed in one of the previous claims,
wherein
an X-ray contrast medium or a dye is incorporated into the plastic of the structural element.
